(19) United States
(12) Patent Application Publication
(10) Pub. No.: US 2004/0265995 A1
(43) Pub. Date: Dec. 30, 2004

(54) sFRP1 and uses thereof

(76) Inventors: Tamara Byk, Kiryat Ono (IL); Ayelet Chajut, Ramat Hasharon (IL)

Correspondence Address: Cooper & Dunham LLP, 1185 Avenue of the Americas, New York, NY, 10036, US

(21) Appl. No.: 10/817,525

(22) Filed: Apr. 1, 2004

Related U.S. Application Data
(60) Provisional application No. 60/459,317, filed on Apr. 1, 2003.

(51) Int. Cl. C12N 5/08

(52) U.S. Cl. 435/366

(57) ABSTRACT

The present invention relates to the use of sFRP1 polypeptide in the induction of stem cell proliferation, and to its function in the treatment of depleted cellular populations.

### PRIORITY

This application claims the benefit of U.S. provisional patent application No. 60/459,317, filed 1, Apr. 2003, which is hereby incorporated by reference in its entirety.

### FIELD OF THE INVENTION

The present invention relates to methods for inducing proliferation of stem cell populations. More specifically, the present invention relates to methods for renewing or expanding populations of Hematopoietic Stem Cells (HSCs) for treating patients suffering from depletion of hematopoietic cell populations.

### SUMMARY OF THE INVENTION

According to the present invention, pharmaceutical compositions that induce stem cell proliferation and uses thereof as medicaments are provided. The present invention describes uses of pharmaceutical compositions comprising sFRP-1 in the treatment of depletion of a cellular population, as well as the implications for use in transplantation and in gene therapy.

### BACKGROUND

Embryogenesis is the fundamental process of differentiation of all tissues from a fertilized egg. During this process, the cells of the developing embryo differentiate and raise their level of commitment, starting out as pluripotent cells, and ending up as fully differentiated mature cells. In mammals, the property of pluripotency is restricted to cells of the early embryo and to tissue-specific stem cells (for review see Smith A. (1998) Curr. Biol. 8(22): R802-R804).

Stem cells are characterized by two main traits: (Morrison, S. J., Shah, N. M. and Anderson, D. J. Regulatory Mechanisms in Stem Cell Biology. Cell 1997, 88: 287-298., Weissman, I. L. Stem Cells: Units of Development, Units of Regeneration, and Units in Evolution. Cell 2000,100: 157-168.).

1. They are pluripotent (they produce daughter cells that can differentiate and give rise to several different cell types).

2. They are self-renewing (they have the capacity to produce daughter cells that maintain the characteristics of the mother stem cell).

Over the past few years, the importance of stem cells for therapy in injury and disease states has been widely recognized. For example, these cells are used to compensate for loss or death of cells or to replace cells with impaired function. The ability to stimulate proliferation and differentiation of stem cells in vivo is crucial for their use in medical and/or therapeutic procedures. Another possibility is to isolate or generate stem cells in culture, and use them for transplantation, such as bone marrow transplantation. In this case, the expansion of the stem cell fraction in the bone marrow, and the induction of proliferation and differentiation after transplantation, can contribute to successful recovery.

Hematopoietic Stem Cells

Hematopoietic stem cells (HSC) are the best characterized stem cells (reviewed in Morrison, S. J. et al. The Biology of Hematopoietic Stem Cells. Annu. Rev. Cell Dev. Biol. 1995, 11:35-71). They persist during lifetime and display the two main characteristics of real stem cells: they are self-renewing, and they are pluripotent. Functionally, HSC are defined by their capacity to repopulate all hematopoietic lineages in marrow ablated animals.

Embryonic Stem Cells

Embryonic stem cells are derived from the totipotent cells of the inner cell mass (ICM) in the early mammalian embryo and are capable of unlimited, undifferentiated proliferation in vitro (Robertson E. J. Embryo-derived Stem Cell Lines. In: Robertson E. J. (ed.) Teratocarcinoma and Embryonic Stem Cells: A Practical Approach. Oxford, JRL Press 1987, 72-112). ES cells are true pluripotent cells as they can differentiate to all cell types.

The ability to obtain fully differentiated cells from the undifferentiated ES cells suggests that in vitro the cells progress through the commitment steps that result in morphological and molecular modifications.

In vitro differentiation of ES cells is extensively used as a model for early mammalian embryogenesis, gene function and development (for reviews see Wobus A. M. and Boheler K. R. (ed) In: Cell Tissue Organs 1999,165:131-245).

Accumulated evidence indicates that ES cells (before and after differentiation) produce growth factors able to induce expansion of hematopoietic stem cells.

Clinical Importance of HSCs

Hematopoietic stem cells are of increasing importance in clinical applications, and are of great importance in bone marrow transplantations. Today, HSC transplantations are performed in a growing spectrum of diseases (Armitage J. O. (1999) in Harrison's Principles of Internal Medicine, 1999); transplantations have been used for many years in cases of leukemia or after the treatment of solid tumors with high-dose chemotherapy or irradiation. There are also reports of the use of HSC transplantations in the treatment of serious blood disorders (Mol Ther 2001 January;3(1):14-23; Int J Hematol 2001 February;73(2):162-9; J Intern Med 2001 April;249(4):379-90; Br J Haematol 2000 March; 108(4):666-78; Haematologica 2000 January;85(1):59-62; J Pediatr Surg. 1993 October;28(10):1232-7), or in autoimmune diseases (nt J Hematol 2001 February;73(2):162-9; J Intern Med 2001 April;249(4):379-90). In all these cases transplantations are either autologous or from MHC matched donors. There is now accumulating evidence that the transplantation of very large doses of HSC can overcome the MHC barrier, the main obstacle for a much larger use of HSC and other transplantations in a variety of additional diseases (Biol Blood Marrow Transplant 1996;2:3-14; Leuk Lymphoma 2001 March;41(1-2):19-34), and induce donor-type tolerance of the host.

Very recent reports have shown that HSC exhibit a much higher degree of cell plasticity than previously believed. If HSCs are injected into damaged tissue or even to the blood stream of animals with tissue damage, they are able to differentiate into skeletal muscle (Ferrari, G. et al. Science 1998,279:1528-1530), hepatocytes (Lagasse, E. et al. Nature Med. 2000,6:1229-1234.), myocardium (Orlic, D. et al. Nature 2001, 410:701-705.), neurons (Mezey, E. et al. Science 2000, 290:1779-1782.; Six, I. et al. Eur J Pharmacol 2003,458:327-328) and participate in the neovascularisation of ischemic myocardium (Kocher, A. A. et al. Nature Med. 2001,7:430436.) retina (Grant, B. et al. Nature Med. 2002, 8:607-612). Thus, there may be many possible clinical uses of HSCs.

The available sources of human HSC are bone marrow, mobilized peripheral blood (MPB), umbilical cord blood and fetal liver. For most transplantations MPB is used. In this case, the donor is treated with G-CSF which induces the mobilization of the HSC from the bone marrow. White cells are then collected from the peripheral blood and HSC purified. Umbilical cord blood is a very good source of multipotent HSC, but the cell number is very limited and can only be used to repopulate the cells of children. Fetal liver 10-14 weeks after gestation is an excellent source of HSC, but rarely available.

The growing need for HSC for transplantation procedures, which often demand higher cell numbers than are actually available, led to intensified efforts to expand HSC in vitro. Expansion protocols leading to cell proliferation without differentiation will not only produce larger stem cell numbers for transplantations, but also improve gene transfer into human HSC.

The main obstacles for HSC transplantation are the lack of MHC-matched donors, the quantity of HSC available and the slow recovery (low neutrophil and platelet counts) of the patients after myeloablative treatment (chemotherapy, irradiation). The first two impediments may be overcome by the ex vivo expansion of HSC, as the transplantation of megadoses of HSC might overcome the graft rejection, and the growing number of frozen umbilical cord blood samples will be a more and more available source of HSC for autologous or allogeneic transplantation.

Ex vivo maintenance and expansion of HSC are also of growing importance for gene therapy protocols. Congenital hematologic diseases can be corrected by the introduction of the intact gene into HSC. The durable expression of the transgene in the differentiated daughter cells has proved to be very low, due to the low gene transfer efficiency to the most primitive repopulating HSC. The ex vivo expansion of HSC without induction of differentiation can overcome this main obstacle to gene therapy in HSC.

sFRP1

The secreted frizzled-related proteins (sFRPs) are approximately 35 kDa in size, and each contains a putative signal sequence, a frizzled-like cysteine-rich domain, and a conserved hydrophilic carboxy-terminal domain. The sFRPs are the products of independent genes. This family of secreted proteins contains a signal peptide necessary for secretion, a cysteine-rich domain (CRD) which is highly homologous to the CRD in frizzled recptors and responsible for Wg binding, and a netrin domain (Rattner et al., Proc. Natl. Acad. Sci. USA, 1997,94: 2859-2863).

The sFRP family members are also known as SARPs—secreted apoptosis related proteins, due to indications that these proteins are involved in the sensitization or desensitization of cells to apoptotic stimuli (Hovsep et al., Proc. Natl. Acad. Sci. USA, 1997,94:13636-13641).

Expression of sFRPs modifies the intracellular levels of β-catenin, suggesting that sFRPs interfere with the Wnt-Frizzled proteins signaling pathway; sFRPs may function in vivo to modulate Wnt signaling, or, alternatively, as novel ligands for as yet unidentified receptors (Rattner et al., Proc. Natl. Acad. Sci. USA, 1997,94: 2859-2863). A sFRP family member, sFRP2, has recently been linked to stimulation of the production of neural progenitors through antagonism of the Wnt pathway (Aubert et al., Nature Biotechnology 20: 1240-1245, 2002).

The Wnt pathway may affect the survival/proliferation of hematopoietic progenitors. It has been suggested that the Wnt pathway is involved in the expansion of HSC: Culturing highly purified mouse bone marrow cells over-expressing β-catenin (a downstream activator of the Wnt pathway) in long-term cultures of HSC expand the transplantable population (Reya T. et al., 2001, Nature 414: 105-111). Interestingly, sFRP1 is a biphasic regulator of Wnt signaling, with high concentrations blocking Wg activity, whereas low concentrations have the opposite effect (Uren A. et al., 2000, J. Biol. Chem. 275:4374-4382).

Additional information concerning the Wnt pathway and hematopoiesis can be found in Constantinescu, J Cell Mol Med. 2003 April-June; 7(2):103-112; Reya, Recent Prog Horm Res. 2003; 58:283-295; Eaves, Nat Immunol. 2003 June;4(6):511-2; Bradbury, Lancet. 2003 May 3;361(9368):1528; Willert et al., Nature. 2003 May 22;423 (6938):448-52; Reya et al., Nature. 2003 May 22; 423(6938):409-14; and Murdoch et al., Proc Natl Acad Sci USA 2003 Mar. 18; 100(6):3422-7.

WO 98/13493 discloses apoptosis-related peptides, including SARPs.

WO 98/54325 discloses a human FRP polypeptide and its uses in cancer therapy.

WO 01/64717 discloses a therapeutic treatment of pulmonary disease, in which sFRPs are inhibited in order to prevent apoptosis.

WO 01/64949 discloses methods and compositions for diagnosing and treating glaucoma, which include sFRP detection or sFRPs.

None of the aforementioned references relate to the use of sFRP1 in the context of stem cell expansion or proliferation.

### BRIEF DESCRIPTION OF THE FIGURE

FIG. 1 Nucleotide (SEQ ID NO:1) and deduced amino acid sequence (SEQ ID NO:2) of sFRP1. The open reading frame comprises consecutive nucleotides from nucleotide 303 to nucleotide 1244 of SEQ ID NO:1.

### DETAILED DESCRIPTION OF THE INVENTION

The present invention relates to methods for renewing or expanding populations of Hematopoietic Stem Cells (HSCs) for treating patients suffering from depletion of hematopoietic cell populations, as discussed above. As shown in the Examples below, the inventors of the present invention have found that the gene sFRP1 is up-regulated in the AGM (aorta-gonado-mesonephros), the first region of long term repopulating HSCs, at the time points when HSCs proliferate most in this region (days 10.5-13.5), whereas it is not up-regulated in the yolk sac and in the fetal liver.

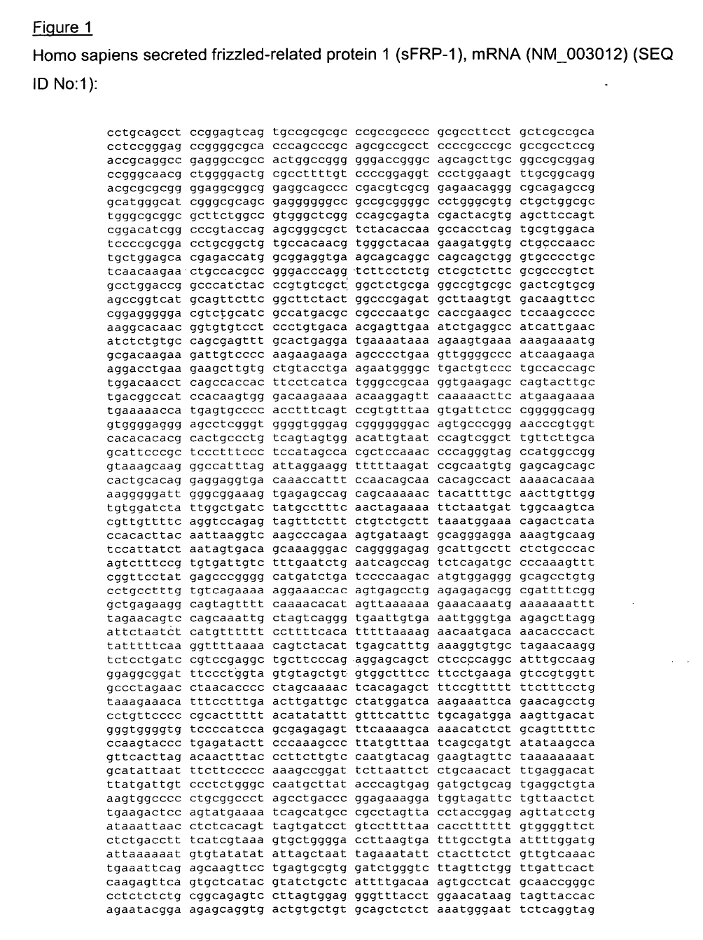

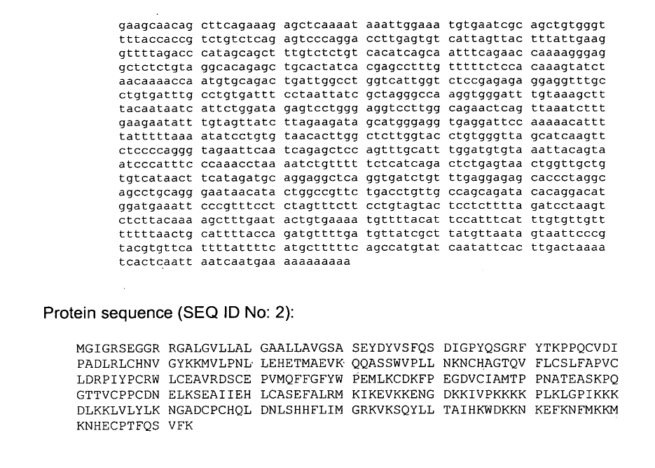

The term “sFRP1”, as used herein refers to the polypeptide of the sFRP1 gene, and is understood to include, for the purposes of the instant invention, the terms “sFRP1”, “SARP” and “SDF5” polypeptides, derived from any organism, preferably man or mice, fragments thereof retaining sFRP1 biological activity, and homologs thereof, preferably having at least 70%, more preferably at least 80%, even more preferably at least 90% or 95% homology thereto. This term is understood to encompass polypeptides resulting from minor alterations in the sFRP1 coding sequence, such as, inter alia, point mutations, deletions and insertions which may cause a difference in a few amino acids between the resultant polypeptide and the naturally occurring sFRP1. Polypeptides encoded by nucleic acid sequences which bind to the sFRP1 coding sequence or genomic sequence under conditions of highly stringent hybridization, which are well-known in the art (for example Ausubel et al., Current Protocols in Molecular Biology, John Wiley and Sons, Baltimore, Md. (1988), updated in 1995 and 1998), are also encompassed by this term. Chemically modified sFRP1 or chemically modified fragments of sFRP1 are also included in the term, so long as the biological activity is retained. The polypeptide sequence of sFRP1 is depicted in FIG. 1 (SEQ ID No: 2). Particular fragments of the sFRP1 polypeptide include amino acids 1-20, 21-40, 41-60, 61-80, 81-100, 101-120, 121-140, 141-160, 161-180, 181-200, 201-220, 221-240 and 241-263 of SEQ ID No: 2. Further particular fragments of the sFRP1 polypeptide include amino acids 10-30, 31-50, 51-70, 71-90, 91-110 111-130, 131-150, 151-170, 171-190, 191-210, 211-230, 231-250 and 251-263 of SEQ ID No: 2.

By “biological effect of sFRP1” or “sFRP1 biological activity” is meant the effect of sFRP1 on stem cells, which may be direct or indirect, and includes, without being bound by theory, the promotion of cell proliferation, be it ex vivo or in vivo, and the ability of sFRP1 to bind to stem cells. The indirect effect includes, but is not limited to, sFRP1 binding to or having an effect on one or several molecules which are involved in a signal transduction cascade resulting in proliferation of stem cells.

By “homolog/homology”, as utilized in the present invention, is meant at least about 70%, preferably at least about 75% homology, advantageously at least about 80% homology, more advantageously at least about 90% homology, even more advantageously at least about 95%, e.g., at least about 97%, about 98%, about 99% or even about 100% homology. The invention also comprehends that these polynucleotides and polypeptides can be used in the same fashion as the herein or aforementioned polynucleotides and polypeptides.

Alternatively or additionally, “homology”, with respect to sequences, can refer to the number of positions with identical nucleotides or amino acid residues, divided by the number of nucleotides or amino acid residues in the shorter of the two sequences, wherein alignment of the two sequences can be determined in accordance with the Wilbur and Lipman algorithm ((1983) Proc. Natl. Acad. Sci. USA 80:726), for instance, using a window size of 20 nucleotides, a word length of 4 nucleotides, and a gap penalty of 4, and computer-assisted analysis and interpretation of the sequence data, including alignment can be conveniently performed using commercially available programs (e.g., Intelligenetics™ Suite, Intelligenetics Inc., Calif.). When RNA sequences are said to be similar, or to have a degree of sequence identity or homology with DNA sequences, thymidine (T) in the DNA sequence is considered equal to uracil (U) in the RNA sequence. RNA sequences within the scope of the invention can be derived from DNA sequences or their complements, by substituting thymidine (T) in the DNA sequence with uracil (U). Additionally or alternatively, amino acid sequence similarity or homology can be determined, for instance, using the BlastP program (Altschul et al., Nucl. Acids Res. 25:3389-3402) and available at NCBI. The following references provide algorithms for comparing the relative identity or homology of amino acid residues of two polypeptides, and additionally, or alternatively, with respect to the foregoing, the teachings in these references can be used for determining percent homology: Smith et al., (1981) Adv. Appl. Math. 2:482-489; Smith et al., (1983) Nucl. Acids Res. 11:2205-2220; Devereux et al., (1984) Nucl. Acids Res. 12:387-395; Feng et al., (1987) J. Molec. Evol. 25:351-360; Higgins et al., (1989) CABIOS 5:151-153; and Thompson et al., (1994) Nucl. Acids Res. 22:4673-4680.

By “polypeptide” is meant a molecule composed of amino acids and the term includes peptides, polypeptides, proteins and peptidomimetics,

The term “Amino acid” refers to a molecule which consists of any one of the 20 naturally occurring amino acids, amino acids which have been chemically modified (see below), or synthetic amino acids.

By “Chemically modified”—when referring to the product of the invention, is meant a product (polypeptide) where at least one of its amino acid residues is modified either by natural processes, such as processing or other post-translational modifications, or by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

The term “expression vector”—refers to vectors that have the ability to incorporate and express heterologous DNA fragments in a foreign cell. Many prokaryotic and eukaryotic expression vectors are known and/or commercially available. Selection of appropriate expression vectors is within the knowledge of those having skill in the art.

The term “alleviation” in the context of disease or illness refers to lessening of symptoms or amelioration of inability to function.

The present invention, in all of its embodiments, provides the possibility of maintaining and/or expanding HSCs and transplanting them to patients suffering from depletion of the hematopoietic cell compartment, so as to alleviate the detrimental effects of depleted cellular populations.

One preferred embodiment of this invention relates to the expansion of cultured stem cells. According to the claimed process, an sFRP1 modulator is administered to cultured stem cells, in a sufficient amount so as to cause proliferation of the stem cells. Said modulator may be an enhancer, which may be a chemical compound. In addition, the modulator may be an expression vector comprising the sFRP1 gene or a fragment thereof, or the sFRP1 polypeptide.

The stem cells which are being induced to proliferate can be, but are not limited to, hematopoietic stem cells or embryonic stem cells.

In the context of stem cell proliferation, the term proliferation refers to growth or multiplication, and is understood to include expansion and renewal of the stem cells (defined, inter alia, by potency to repopulate irradiated mice or humans).

A “modulator” is any molecule that is capable of modulation, i.e. that either increases (promotes) or decreases (prevents). The term is understood to include partial or full inhibition, stimulation and enhancement.

In the context of the present invention, by “inhibitor” and “enhancer” is meant any molecule that can inhibit or enhance the biological activity of sFRP1, respectively.

The terms “chemical compound”, “small molecule”, “chemical molecule” “small chemical molecule” and “small chemical compound” are used interchangeably herein and are understood to refer to chemical moieties of any particular type which may be synthetically produced or obtained from natural sources and typically have a molecular weight of less than 2000 daltons, more preferably less than 1000 daltons or even less than 600 daltons.

The term “antibody”—refers to IgG, IgM, IgD, IgA, and IgE antibody, inter alia. The definition includes polyclonal antibodies or monoclonal antibodies. This term refers to whole antibodies or fragments of the antibodies comprising the antigen-binding domain of the anti-GPCRV product antibodies, e.g. antibodies without the Fc portion, single chain antibodies, fragments consisting of essentially only the variable, antigen-binding domain of the antibody, etc. The term “antibody” may also refer to antibodies against nucleic acid sequences obtained by cDNA vaccination.

Antibody fragments retain the ability to selectively bind with their antigen or receptor and are exemplified as follows, inter alia:

(1) Fab, the fragment which contains a monovalent antigen-binding fragment of an antibody molecule can be produced by digestion of whole antibody with the enzyme papain to yield a light chain and a portion of the heavy chain;

(2) (Fab′)₂, the fragment of the antibody that can be obtained by treating whole antibody with the enzyme pepsin without subsequent reduction;

F(ab′₂) is a dimer of two Fab fragments held together by two disulfide bonds;

(3) Fv, defined as a genetically engineered fragment containing the variable region of the light chain and the variable region of the heavy chain expressed as two chains; and

(4) Single chain antibody (SCA), defined as a genetically engineered molecule containing the variable region of the light chain and the variable region of the heavy chain linked by a suitable polypeptide linker as a genetically fused single chain molecule.

Details on how to prepare all types of antibodies are provided in Example 8 below. An additional embodiment of the present invention concerns a process for inducing proliferation of stem cells comprising administering to cultured stem cells sFRP1 in a sufficient amount to cause proliferation of the stem cells.

The stem cells which are being induced to proliferate can be, but are not limited to, hematopoietic stem cells or embryonic stem cells.

In another embodiment, the present invention provides a process for inducing proliferation of stem cells comprising culturing the stem cells with a second type of cells wherein the second type of cells express or overexpress sFRP1; in addition, sFRP1 may be administered to the stem cells.

By “culturing with” is meant growing the stem cells in culture with the second type of cells, preferably on the second type of cells.

The second type of cells may over-express sFRP1 (methods for causing cells to over-express the polypeptide of a certain gene are well known in the art), and are preferably stromal cells.

Stromal cells provide a bed for closely associated hematopoietic cells in the bone marrow (reviewed in Dorshkind: “Regulation of hemopoiesis by bone marrow stromal cells and their products”, Annu Rev Immunol. 1990;8:111-37), where they are in close contact with hematopoietic cells through adhesion molecules which transmit them together with secreted molecules proliferation and differentiation signals.

Stromal cells can be, but are not limited to, non-hematopoietic bone marrow cells of mesenchymal origin lacking the general leukocyte marker CD45. They may be of variable morphologic types including preadipocytes, adipocytes, smooth-muscle-like, fibroblastoid, endotheloid and epitheloid. Additionally, they may express specific markers like stro-1 and adhesion molecules directly involved in the binding of hematopoietic cells, such as VLA4, N-CAM and V-CAM.

Another embodiment of the present invention concerns a method for treating a patient suffering from depletion of a cellular population, comprising administering to the patient stem cells that have been expanded according to the methods of the present invention. The patient may be suffering from depletion of a cellular population as a result of a disease or a disease treatment; the disease may be, inter alia, cancer, a blood disorder or an auto-immune disease; the treatment may comprise chemotherapy or radiotherapy.

In addition, the present invention provides a method for treating a patient suffering from depletion of a cellular population comprising administering to the patient a pharmaceutical composition comprising an sFRP1 modulator, further comprising a pharmaceutically acceptable carrier, in a dosage sufficient to induce proliferation of a cellular population. Said modulator may be an enhancer, which may be a chemical compound. In addition, the modulator may be an expression vector comprising the sFRP1 gene or a fragment thereof, or the sFRP1 polypeptide.

The present invention also provides a method for treating a patient suffering from depletion of a cellular population comprising administering to the patient, a pharmaceutical composition comprising sFRP1, further comprising a pharmaceutically acceptable carrier, in a dosage sufficient to induce proliferation of a cellular population.

The above methods may be employed to treat a patient who is suffering from depletion of a cellular population as a result of a disease or a disease treatment; the disease may be, inter alia, cancer, a blood disorder or an auto-immune disease; the treatment may comprise chemotherapy or radiotherapy.

By “depletion of a cellular population” is meant that the patient, as a consequence of disease or adverse effects of certain disease treatment, no longer has a sufficient amount of a certain type of cells in order to function as before the onset of the disease or without pain. The stem cells administered to the patient may have the capacity to replace the type of cells which have been depleted in the patient, and in so doing, alleviate the symptoms associated with the depletion of a cellular population.

The term “cellular population” refers to a population of more then one cell, wherein all the cells are of the same type. Cellular populations include, but are not limited to, populations of skeletal muscle cells, myocardial cells, bone marrow cells, nervous cells, blood cells, hematopoietic stem cells, embryonic stem cells and stromal cells.

By “chemotherapy” is meant treatment with a chemotherapeutic drug, such as, inter alia: etoposide, 5-FU (5-fluorouracil), cis-platinum, doxorubicin, a vinca alkaloid, vincristine, vinblastine, vinorelbine, taxol, cyclophosphamide, ifosfamide, chlorambucil, busulfan, mechlorethamine, mitomycin, dacarbazine, carboplatinum, thiotepa, daunorubicin, idarubicin, mitoxantrone, bleomycin, esperamicin A1, dactinomycin, plicamycin, carmustine, lomustine, tauromustine, streptozocin, melphalan, dactinomycin, procarbazine, dexamethasone, prednisone, 2-chlorodeoxyadenosine, cytarabine, docetaxel, fludarabine, gemcitabine, herceptin, hydroxyurea, irinotecan, methotrexate, oxaliplatin, rituxin, semustine, tomudex and topotecan, and chemotherapeutically active analogs of these drugs.

Depletion of a cellular population as a direct or indirect (through side-effects of treatment methods for said illness, for example) result of any other illness, can also be treated by administration to the patient of stem cells that have been expanded using the aforementioned claimed methods.

The claimed method of treatment offers the possibility of transplanting very large numbers of stem cells to the patient. The large number of stem cells can overcome host-rejection, and the stem cells can differentiate into the cell type in which the patient has been depleted as a result of the disease.

The present invention further provides a pharmaceutical composition comprising an sFRP1 modulator further comprising a pharmaceutically acceptable carrier. Said modulator may be an enhancer, which may be a chemical compound. In addition, the modulator may be an expression vector comprising the sFRP1 gene or a fragment thereof, or the sFRP1 polypeptide.

In addition, the present invention provides a pharmaceutical composition comprising sFRP1 further comprising a pharmaceutically acceptable carrier.

In addition, the above pharmaceutical compositions may be formulated in a dosage sufficient to induce proliferation of a cellular population. For additional information on dosage and formulation see Example 5.

An additional embodiment of the present invention provides for the use of any one of the above pharmaceutical compositions as a medicament. In particular, this embodiment provides for the use of a pharmaceutical composition comprising sFRP1 or an sFRP1 modulator as a medicament. Said modulator may be an enhancer, which may be a chemical compound. In addition, the modulator may be a vector comprising the sFRP1 gene or a fragment thereof. In a preferred embodiment, the pharmaceutical composition being used as a medicament causes proliferation of a cellular population. An additional embodiment provides for sFRP1 for use as a medicament, and for the use of sFRP1 for expansion of stem cells, or for use of sFRP1 in the preparation of a composition for expansion of stem cells.

The present invention also provides a use of sFRP1 or an expression vector comprising the sFRP1 gene or a fragment thereof in the preparation of a medicament for the treatment of depletion of a cellular population, as a result of a disease or a disease treatment; the disease may be, inter alia, cancer, a blood disorder or an auto-immune disease; the treatment may comprise chemotherapy or radiotherapy.

It is understood that, in the context of an additional embodiment of the present invention, it may be beneficial to treat a patient with an sFRP1 inhibitor. Said inhibitor may be, inter alia, an antibody, an antisense molecule or a vector encoding an antisense molecule, an siRNA molecule or a vector encoding an siRNA molecule, or a chemical compound.

An additional embodiment of the present invention provides a process for identifying a compound which induces stem cell proliferation by modulation of sFRP1 comprising:

(a) measuring the proliferative activity of the human sFRP1 polypeptide;

(b) contacting said polypeptide with said compound; and

(c) determining whether the activity of said polypeptide is affected by said compound.

This embodiment further provides a process of preparing a pharmaceutical composition which comprises the steps of:

(a) obtaining a compound by the above process; and

(b) admixing said compound with a pharmaceutically acceptable excipient.

In addition, a process for identifying a compound which induces stem cell proliferation by modulation of sFRP1 is provided, comprising:

a) measuring the binding of sFRP1 to a species with which it interacts in vivo;

b) contacting sFRP1 with said compound; and

c) determining whether the activity of sFRP1 is affected by said compound.

A kit for identifying a compound which induces stem cell proliferation is also provided, comprising:

(a) sFRP1;

(b) a species with which sFRP1 interacts in vivo;

(c) means for measuring said interaction; and

(d) means for determining whether the binding of sFRP1 to the species is affected by said compound.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Example 1

Methods

Mouse General Development (MGD) Microarray

The MGD microarray is imprinted with cDNAs derived from Embryoid Bodies at late spontaneous differentiation stages, teratocarcinomas and mouse embryos.

Differentiated ES Cells (Embryoid Bodies—EBs)

1. Differentiation in Suspension:

Large populations of undifferentiated mouse ES cells (129/Sv) are synchronously induced to start differentiation in culture by growing the cells in suspension, using bacteriological petri dishes. The cells adhere to each other and form small three-dimensional structures within 24 hours. Four to five days later, almost 100% of the aggregates exhibit endoderm formation (termed simple embryonic bodies—SEBs). SEBs occur after an additional incubation period of 8-10 days. By this time, a high percentage of the EBs develop fluid filled cavities accompanied by formation of ectoderm-like cells.

2. Differentiation on a Substrate

SEBs formed after 4 days in suspension culture are passed on gelatinized tissue culture plates. The EBs attach to the plate surface by the outgrowth of endodermal cells. Continued culture of these aggregates gives rise to an array of cell phenotypes like nerve, muscle, cartilage, hematopoietic cells and more.

Teratocarcinomas

When ES cells are injected subcutanously into nude mice, tumors are readily formed. These tumors contain differentiated tissues of all kinds, similar to those in the developing embryo, but in an unorganized pattern.

Mouse Embryos

Different days of development in the mouse represent different differentiation stages. First HSCs can be detected at 10.5 dpc (days post coitus).

Library Preparation

cDNA was prepared from RNA collected from differentiated embryonic bodies (EBs) at the indicated time points, mouse teratocarcinomas 16, 32 and 40 days after injection and mouse embryos at 9.5, 11.5, 13.5, 15.5 and 17.5 dpc (table 1).

TABLE 1
MGD Chip design

| | Days after | |
|---|---|---|
| | differentiation | Time points |
| Cells | induction | (total) |
| EBs, | 11-14 | Once in 2 days |
| Differentiation in | 15-21 | Once in 3 days |
| suspension | | |
| EBs, Attached | 11-14 | Once in 2 days |
| Differentiation | 15-21 | Once in 3 days |
| Mouse | | 16, 32 40 days |
| Teratocarcinomas | | after injection |
| Mouse Embryos | | Days 9.5, 11.5, |
| | | 13.5, 15.5, 17.5 |

Three libraries were prepared according to the methods disclosed in co-assigned PCT publication WO 02/45472 (“Prime and Kill”), as follows:

from the spontaneously differentiated EBs collected at the time points indicated in table 1(MLE),

from the teratocarcinomas collected at the indicated time points (MTC) and

from the mouse embryos collected at the indicated time points (MEB).

In addition, cDNAs representing regulated genes known form the literature (up- or down-regulated and indifferent during ES cells differentiation) were printed on the microarray as controls.

Mouse Hematopoietic (MHB) Microarray

This microarray is imprinted with cDNAs from different sub-populations of primitive hematopoietic cells.

The hematopoieitc system is organized in a pyramide-like manner, with the most primitive, pluripotent and self-renewing stem cells on the top. These cells are functionally defined by their ability to long-term repopulate (for at least six months) the bone marrow of lethally irradiated recipient mice and to produce all the cell lineages in the blood (LTR-HSC).

The less primitive short-term-repopulating cells are still pluripotent, but do not self-renew. They repopulate irradiated mice up to two months.

Short term repopulating cells differentiate into more multipotent progenitors with more restricted differentiation capacities. These further differentiate to committed progenitors and finally differentiated, mature cells.

Lin-Neg. Cell Fraction (Fraction 1)

The lin-neg. cell fraction contains all bone marrow cells not expressing any differentiation marker (i.e. lineage marker) of hematopoietic cells. This cell fraction represents a mixed cell population containing all the primitive hematopoietic stem cells (long- and short term repopulating cells as well as very early progenitors).

Lin-Neg. Cell Fraction “on Stroma” (Fraction 2)

In the bone marrow, HSC are in close interaction with stromal cells. Stromal cells regulate HSC proliferation and differentiation through growth factors as well as direct cell-cell contact. They therefore influence the gene expression pattern of HSCs. To mimic this type of interaction, HSC were incubated for 12 hours on a bone-marrow derived stromal cell line (pre-adipocyes, FBMD-1) and thereafter re-separated from the stromal cells by fluorescence-activated cell sorting (FACS).

“LTR-HSC” (Fraction 3)

The long term repopulating HSC cell fraction was defined by the expression of the following antigens: Stem cell antigen-1 (Sca-1)-positive/stem cell factor-receptor (c-kit)-positive, lineage-negative/CD34-negative (Sca-1⁺/lin⁻/c-kit⁺/CD34⁻, Osawa M. et al., (1996) Science 273:342-345). This population was isolated from total mouse bone marrow by four-color-FACS.

Library Preparation

The array was imprinted with three different types of libraries; some were prepared according to the methods disclosed in co-assigned PCT WO 01/75180 publication (“SDGI”). The libraries were the following:

1. From each of the fractions a full-length library was produced.

2. Two SDGI libraries were created: from a pool of fractions land 2 and from fraction 3.

3. A gene expression Fingerprint (GEF) differential library enriched in cDNA fragments characterizing the LTR-HSC cell population (Zinovyeva M. V. et al., (2000) Exp. Hematol. 28:318-334).

In addition, control cDNAs of known genes from the literature were printed on the microarray.

Mouse Stroma (MST) Chip

This chip is imprinted with cDNAs derived from stromal cell lines subjected to different treatments, primary total bone marrow cultures subjected to different treatments and fetal livers.

Fetal Livers

The fetal liver is the main hematopoietic organ in the fetus from E13 until close to birth, when the HSC migrate to the bone marrow. HSC can be detected in the fetal liver already from E11, when they start migrating from the AGM and the yolk sac (Dzierzak E et al. (1998) Immunol. Today 19:228-236). The fetal liver must therefore present an ideal environment containing HSC supporting stroma. Several HSC supporting cell lines were deduced from fetal liver (Charbord P. et al., (2002) Exp. Hematol. 30:1202-1210).

Primary Bone Marrow Cultures

Total bone marrow grown at high fetal calf and horse serum concentrations produces an adherent “stromal” cell layer and long term hematopoiesis. The adherent cell layer plays a role in the maintenance of the hematopoietic stem cells, which can stay for months in a quiescent state under the stromal cell layer, until they start proliferating and differentiating. Such “Dexter-type” cultures produce preferentially myeloid cells and can be maintained for months (Dexter T. M. et al., (1976) J. Cell. Physiol.91:335-344). The use of low fetal calf serum concentration and poor medium in such total bone marrow cultures leads also to the development of a stromal feeder layer, which supports preferentially the development of pre-B and B cells (Whitlock C. A. and Wifte O. N., (1982) Proc. Natl. Acad. Sci. USA 79:3608-3612).

Stromal Cell Lines

Many stromal cell lines were isolated from primary “Dexter-type” bone marrow cultures. These lines support maintenance of HSC for several weeks. MS-5, FBMD-1 and 14F1.1 are pre-adipocyte-type stromal cell lines well defined in the literature (Itoh K. et al., (1989) Exp. Hematol. 17:145-153; Breems D. A. et al., (1994) Leukemia 8:1095-1104; Zipori D. et al., (1985) Blood 66:447-455). The maintenance potential of stromal cell lines is strongly dependent on the presence of high fetal calf and horse serum concentrations. Hydrocortisone and LIF and, in certain cases, bFGF and hypoxia, improve the maintenance potential of stromal cell lines. G-CSF and cyclophosphamide induce in vivo the mobilization of HSC from the bone marrow to the blood and influence therefore the cell-cell interaction between HSC and stroma. γ-irradiation is used for myeloablation of hosts to be repopulated and was shown to substantially increase the homing efficiency of injected HSC, in correlation with the up-regulation of several genes in the stroma of the bone marrow (for example, SDF).

Library Preparation

The array was imprinted with three different types of libraries, all prepared by the “prime and kill” method described above:

MFL: Mouse fetal livers at developmental days 9.5, 10.5, 11.5 and 12.5. The redundancy of this library was initially relatively high (42%) and was further reduced by screening it with probes for the three most redundant genes, and only negative clones were selected.

MBC: Dexter-type primary bone marrow cultures grown with or without hydrocortisone, LIF, G-CSF, subjected to hypoxia or gamma-irradiated, and Whitlock-Witte cultures.

MSL: Mouse stromal cell lines (MS-5, FBMD-1 and 14F1.1 subjected to treatments of hydrocortisone, LIF, FCS+horse serum, cyclophosphamide, G-CSF for 1 or 7 days and after hypoxia or gamma-irradiation.

Probe Design and Hybridizations

Common Normalizing Probe

In order to achieve meaningful differentials of expression profiles it is of importance to normalize the signals obtained in each hybridization. Therefore, in each hybridization, in addition to the probe of interest which is labeled with one fluorescent dye, a second reference probe is added, labeled with a second dye. While the test probe varies from one hybridization to another, the reference probe, also called “common normalizing probe”, is invariant and permits the normalization of the hybridization signals and herewith the comparison between two independent hybridizations.

For the hybridizations on the MHB microarray using different HSC subfractions as specific probes, the lin-cell fraction was used as the common normalizing probe (biologically relevant common probe).

For the hybridizations on the MGD microarray, the common normalizing probe used was a mixture of RNA from undifferentiated ES cells (6 parts), and one part of each of the libraries printed on the microarray (Embryoid Bodies at late differentiation stages, teratocarcinomas, mouse embryos; biologically irrelevant common probe). For the MST microarray, the common probe used was a 1:1 mix of RNA derived from the three different stromal lines after different treatments, and rat brain RNA (biologically irrelevant common probe).

Specific Probes

One set was prepared from MS-5, FBMD-1 and MBA14F1.1 cells (hematopoiesis supporting adipocyte-like stromal cell lines) and MBA13 cells (non-supporting cell line). The cell lines were treated with hydrocortisone, horse serum, hypoxia and, for MBA13 and MBA14F1.1, were collected at subconfluent state. This set was used for the hybridizations on the MGD and the MST microarrays.

A second set of probes was prepared from different sub-fractions of the lin-negative cell population: Sca-1⁺/lin⁻/c-kit⁻/CD34⁻, Sca-1⁺/lin⁻/c-kit⁺/CD34⁻, Sca-1⁺/lin⁻/c-kit⁺/CD34⁺, Sca-1⁺/lin⁻/c-kit⁻/CD34⁺, and the total lin⁻ fraction itself. This set was used for the hybridizations on the MHB microarray.

In addition, “membrane-bound” mRNA probes, in which mRNA for membranal and secreted proteins are enriched, were prepared from undifferentiated ES cells as well as MS-5 and FBMD-1 cells. This is in order to identify a maximum of cDNAs coding for membranal and secreted proteins on all the three microarrays.

Statistical and Bioinformatic Analysis of the Hybridization Results

Statistical algorithms adapted by the inventors for the study of gene expression patterns along the time course of a specific treatment were used. This method enabled the inventors to build clusters of genes, the expression pattern of which correlates with the regulation of the potency of the stromal cell line to support hematopoiesis.

Following sequencing, sequence analysis tools were used for annotation and extension mining of sequence information. Those tools include Phred phrap, BLAST, and other public databases, along with proprietary applications.

Validation of Selected Candidate Genes

Initial verification of the hybridization results was carried out by RT-PCR on mRNA extracted from MS-5, FBMD-1 and 14F1.1 cells after different treatments. For candidates confirming the hybridization results and/or with an interesting expression profile in the different cell lines after different treatments (i.e.up- or down-regulated in a line or treatment known to support in vitro hematopoiesis), in situ hybridization on mouse embryos at early stages was performed to find expression in the AGM, expression specificity in this region and possible expression in the liver and yolk sac. Furthermore, adult bone marrow, thymus and spleen were analyzed.

Candidate genes were then expressed in stromal cell lines and the effect of the transgene on stem cell maintenance and expansion tested in LTC-IC and CFAC assays in vitro. Then, the effect of the over-expressed protein was tested by injection of HSC cultured on stroma over-expressing the candidate gene to irradiated animals for repopulation assays.

Candidate genes showing a positive effect on the in vitro maintenance of HSC in these assays, are produced as recombinant proteins and tested in vitro directly on the HSC and in vivo by injection to transplanted animals.

The sFRP1 gene was identified by several of the above methods, as described in Example 3.

### Example 2

Analysis of HSCs

Several different in vivo and in-vitro assays were utilized in order to estimate the presence, amount and differentiation state of the HSCs (for review see Domen J. & Weissman I.L. Mol. Med. Today 1999, 5: 201-208).

In Vivo Assays:

1. Long/short term reconstitution of mice—A defined number of cells of interest, which can be un-separated, e.g., bone marrow cells or any purified cell population, is injected to irradiated mice. The contribution of the donor cells to the total peripheral blood or bone marrow of the host is assayed after a period of 2 months (STR-HSC) or after a period of over 6 months (LTR-HSC). To quantify more precisely the amount of stem cells, competitive repopulation units (CRU) are estimated by injecting limiting numbers of donor-type cells together with a standard number of host-type total bone marrow cells. (Szilvassy, S. J. et al. Proc. Natl. Acad. Sci. USA 1990, 87: 8736-8740). The frequency of CRU is then calculated from the proportion of negative mice (showing less than 3% donor-type cells in the bone-marrow) by the method of maximal likelihood for each injected cell number (Taswell, C. J. Immunol. 1981,126: 1614-1619).

2. Colony-forming-units-spleen (CFU-S)—In this assay, lethally irradiated mice are injected with a HSC population of interest and colonies formed on the spleen are counted after 7 and 14 days. This assay is of limited interest for stem cells, as also early progenitors can form similar colonies.

In Vitro Assays:

1. Long-term-culture-initiating cell (LTC-IC)—The cell population to be tested is plated on stroma feeder cells and hematopoietic progenitor cells are quantified after 5 weeks. This in vitro assay reveals a cell population closest to the stem cells identified in repopulation assays, but taking into consideration their higher frequency and the relative facility of LTC-ICs transduced with viral vectors, they seem to be less primitive cells than STR-HSCs.

2. Cobblestone area forming cell (CAFC)—The cell population of interest is plated on stroma cells and the cultures scored for light-dense colonies called “cobble-stone areas” 7-35 days after plating. This assay reveals a population resembling LTC-IC, but is much less quantitative.

3. Cobblestone area forming cell-limiting dilution (CAFC-LD) The cell population to be tested is plated on stromal cells in a limiting dilution mode by varying the number of inoculated cells per culture over a wide range. Cultures containing no “cobblestone area” were counted each week and from the percentage of negative cultures in relation to the number of plated cells the number of CAFC can were accurately determined. CAFC producing late appearing cobblestone areas (28-35 days) are considered to represent the more primitive stem cells, whereas early appearing cobblestone areas (7-14 days) reflect the presence of less primitive stem and progenitor cells. (Breems D. A., et al. Leukemia 1994, 8: 1095-1104).

Isolation and Culturing of HSCs ex Vivo

Isolation: Sca-1⁺ cells were separated form total bone marrow cells by Ficoll gradient and Sca-1⁺-cell labeling by anti-Sca-1 antibodies coupled to microbeads and passage through a magnetic column. The positive population, remaining in the column was collected. Cells were then labeled for lineage markers, Sca-1 and c-kit and Sca-1⁺/lin⁻/c-kit⁺ cells were sorted by FACS.

Culturing: isolated cells were cultured in the presence of stromal cells, in the following medium: alpha-MEM medium containing 12.5% FCS (Stem Cell Inc.), 12.5% HS (Stem Cell Inc.), 100 U/ml Penicillin, 0.1 mM β-mercaptoethanol. Half the medium was changed once weekly.

### Example 3

### Experimental Results

Expression Pattern

Hybridization on the MGD DNA array using probes from different hematopoietic organs in the mouse embryo (as detailed in Example 1) was performed and the following results were obtained for sFRP1:

sFRP1 is up-regulated in the AGM from the moment HSCs begin to appear (10.5 late dpc), and stays up-regulated during the in vivo (11.5, 12.5 and 13.5) and ex vivo (10.5 and 12.5 cultured) expansion of HSCs. It is AGM specific, which might also indicate that it plays a role in HSC generation.

TABLE 2
AGM

| | | 10.5 | 10.5 | | 11.5 | | 12.5 | |
|---|---|---|---|---|---|---|---|---|
| 9.5 | 10.5 | late | cult | 11.5 | cult | 12.5 | cult | 13.5 |
| 1 | 1.4 | 1.7 | 1.5 | 2.4 | 1.1 | 1.9 | 1.4 | 1.9 |

Fetal Liver

| | 10.5 | | 11.5 | | |
|---|---|---|---|---|---|
| 10.5 | late | 11.5 | cult | 12.5 | 13.5 |
| 1.5 | 1.3 | −1.1 | −1.3 | −1.3 | −1.8 |

Yolk Sac

| | | 10.5 | | 11.5 | | 12.5 |
|---|---|---|---|---|---|---|
| 9.5 | 10.5 | late | 11.5 | cult | 12.5 | cult |
| −1.8 | −1.9 | −1.6 | −1.3 | −1.3 | −1.6 | −1.3 |

In stromal cell lines, sFRP1 is expressed most strongly in FBMD-1 cells (the highest supporting line—a result which has been confirmed in RT-PCR assays, see below).

TABLE 3
MS-5

| | HS- | HS- | HC- | HC- | |
|---|---|---|---|---|---|
| cont | 1d | 7d | 1d | 7d | Hypox |
| 1 | 1 | −1.1 | 1.2 | −1 | 1 |

FBMD-1

| | HS- | HS- | HC- | HC- | |
|---|---|---|---|---|---|
| cont | 1d | 7d | 1d | 7d | hypox |
| 2.3 | 1.7 | 1.4 | 2.2 | 2.4 | 2.3 |

14F1.1

| | HS- | HS- | HC- | HC- | | |
|---|---|---|---|---|---|---|
| Cont | 1d | 7d | 1d | 7d | hypox | sub |
| 1.3 | 1.4 | 1 | 1.2 | 1.1 | 1.1 | 1.2 |

MBA13

| | HS- | HC- | |
|---|---|---|---|
| cont | 7d | 7d | Sub |
| 1 | 1 | 1.3 | 1.3 |

RT-PCR

RT-PCR was performed on three stromal cell lines after treatment with reagents known to change their capacity to support HSC long-term maintenance. In MS-5 and FBMD-1, sFRP1 was regulated after different treatments, and the hybridization results for hydrocortisone and horse serum in FBMD-1 were confirmed. In MS-5 cells hydrocortisone strongly up-regulates sFRP1 expression. This was not observed in the hybridizations, because in MS-5 cells it is expressed at much lower levels, probably below the sensibility of this assay (see also below, Table comparing sFRP1 expression level in the different cell lines). As observed in the hybridizations, no expression regulation of sFRP1 was observed in MBA14F1.1 cells.

TABLE 4
Effect of different treatments on the expression of sFRP1
in stromal cell lines.

| | MS-5 | FBMD-1 | 14F1.1 |
|---|---|---|---|
| Control 1d | 1.00 | 1.00 | 1.00 |
| Control 7d | 1.83 | 2.18 | 0.38 |
| bFGF 1d | N.D. | 0.39 | 0.53 |
| bFGF 7d | 1.96 | 2.32 | 0.60 |
| Hydrocortisone 1d | 20.52 | 4.54 | 1.15 |
| Hydrocortisone 7d | 2.36 | 2.73 | 0.48 |
| LIF 1d | 4.44 | 8.28 | 1.15 |
| LIF 7d | 1.05 | 3.46 | 0.38 |
| Horse Serum 1d | 3.61 | 0.81 | 0.95 |
| Horse Serum 7d | 0.49 | 1.80 | 0.29 |
| Cyclophosph. 1d | N.D. | 1.97 | 0.51 |
| Cyclophosph. 7d | 1.00 | 1.90 | 0.20 |
| G-CSF 1d | 10.07 | 2.13 | 0.50 |
| G-CSF 7d | 0.52 | 1.10 | 0.51 |
| Hypoxia (16 hrs)*** | 0.76 | 0.19 | 0.41 |
| Irradiation (300 rad) | 9.29 | 3.60 | 1.57 |

The numbers express the factor of RNA level compared to the corresponding control cells 1 day, and were normalized with the GAPDH values obtained for each sample.
**The values for hypoxia are probably higher taking into consideration that GAPDH is up-regulated under hypoxia.

There was a strong difference in the expression level of sFRP1 in the different stromal cell lines:

TABLE 5

| MS-5 | FBMD-1 | 14F1.1 | MBA13 |
|---|---|---|---|
| 0 | ++++ | 0 | +++++++++ |

+ up-regulated
− down-regulated 0 unchanged

In Situ Hybridization

Hybridization to antisense probe resulted in a hybridization signal that was observed in 11.5 and 12.5 dpc sections. At 11.5 dpc the hybridization signal locates to mesenchymal cells at different locations within the embryonic body including the tissue surrounding the aorta and cardinal veins, the mesenchyme of the body wall, the developing diaphragm and head. A strong hybridization signal also locates to the brain.

At 12.5 dpc a hybridization signal was associated with mesenchymal cells localized in different parts of the embryonic body including, in addition to areas described at 11.5 dpc, condensations of head mesenchyme involved in development of the inner ear and nasal capsule. Endothelial cells within loose mesenchyme display a clear hybridization signal.

In addition, strong expression was detectable in some areas of the brain.

No hybridization signal could be seen in liver but mesenchyme associated with mesonephric tubules (mesonephors are almost completely regressed at this stage) shows strong expression. Interestingly, metanephric expression is confined to the capsule. This difference in the pattern of expression between meso- and metanephros may point to the involvement of the gene product into AGM associated generation of hematopoietic stem cells. Certainly, no expression was detected in embryonic liver.

Adult expression. No signal was detected on multiblock sections hybridized to T3 and T7 probes. Weak or no hybridization signal was detectable on bone sections. This signal was associated with osteoprogenitors and osteoblasts in cancellous bone in both intact and irradiated samples.

In Vitro and in Vivo Validation

1. sFRP1-flag was cloned into the retroviral vector PLNCX, MS-5 and FBMD-1 cells were infected and over-expressing cells selected to produce stably expressing lines. For control, MS-5 and FBMD-1 cells were infected with the empty retroviral vector (mock-infected).

2. MS-5 cells and FBMD-1 stably over-expressing the full length sFRP1-flag protein (shown by Western blot analysis) are used in LTC-IC assays: Long term cultures are performed with sorted sca-1⁺/lin⁻/c-kit⁺ cells at the presence of hydrocortisone on sFRP1 over-expressing and control (mock-infected) cells.

3. In vivo assays are performed by maintenance of the cells during one or two weeks on sFRP-1 over-expressing MS-5 or FBMD-1 cells. HSC and stromal cells are then collected and injected to irradiated mice for repopulation assay. Control cells are maintained and assayed identically, with the exception of being grown on stromal cells that do not over-express sFRP-1⁻.

### Example 4

Gene Therapy

By gene therapy as used herein refers to the transfer of genetic material (e.g DNA or RNA) of interest into a host to treat or prevent a genetic or acquired disease or condition phenotype. The genetic material of interest encodes a product (e.g. a protein, polypeptide, peptide, functional RNA, antisense fragment, GIE) the production of which in vivo is desired. For example, the genetic material of interest can encode a hormone, receptor, enzyme, polypeptide or peptide of therapeutic value. Alternatively, the genetic material of interest may encode a suicide gene. For a review see, in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject rather the genetic material to be transferred is introduced into the cells of the recipient organism in situ, that is within the recipient. In an alternative embodiment, if the host gene is defective, the gene is repaired in situ (Culver, 1998. Site-Directed recombination for repair of mutations in the human ADA gene. (Abstract) Antisense DNA & RNA based therapeutics, February, 1998, Coronado, Calif.). These genetically altered cells have been shown to express the transfected genetic material in situ.

The gene expression vehicle is capable of delivery/transfer of heterologous nucleic acid into a host cell. The expression vehicle can include elements to control targeting, expression and transcription of the nucleic acid in a cell selective manner as is known in the art. It should be noted that often the 5′UTR and/or 3′UTR of the gene can be replaced by the 5′UTR and/or 3′UTR of the expression vehicle. Therefore as used herein the expression vehicle can, as needed, not include the 5′UTR and/or 3′UTR of the actual gene to be transferred and only include the specific amino acid coding region.

The expression vehicle can include a promotor for controlling transcription of the heterologous material and can be either a constitutive or inducible promotor to allow selective transcription. Enhancers that can be required to obtain necessary transcription levels can optionally be included. Enhancers are generally any non-translated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods. Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector for introducing and expressing recombinant sequences is the adenovirus derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject. Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation can not occur.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention depends on desired cell type to be targeted and is known to those skilled in the art. For example, if breast cancer is to be treated then a vector specific for such epithelial cells are used. Likewise, if diseases or pathological conditions of the hematopoietic system are to be treated, then a viral vector that is specific for blood cells and their precursors, preferably for the specific type of hematopoietic cell, is used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed do not contain a genome and therefore cannot proceed through subsequent rounds of infection. The specific type of vector depends upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, and administration can also be performed by, for example, intravenous or subcutaneous injection into the subject. Injection of the viral vectors into a spinal fluid can also be used as a mode of administration, especially in the case of neuro-degenerative diseases. Following injection, the viral vectors circulate until they recognize host cells with the appropriate target specificity for infection.

An alternate mode of administration can be by direct inoculation locally at the site of the disease or pathological condition or by inoculation into the vascular system supplying the site with nutrients or into the spinal fluid. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the non-viral vectors described above into recipient cells within the inoculated area. Such transfection vehicles are known by one skilled within the art.

### Example 5

Pharmacology and Drug Delivery

The medicament or pharmaceutical composition of the present invention is administered and dosed in accordance with good medical practice, taking into account the clinical condition of the individual patient, the disease to be treated, the site and method of administration, scheduling of administration, patient age, sex, body weight and other factors known to medical practitioners. The pharmaceutically “sufficient dose” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

The treatment generally has a length proportional to the length of the disease process and drug effectiveness and the patient species being treated. It is noted that humans are treated generally longer than the mice or other experimental animals exemplified herein.

The medicament or pharmaceutical composition of the present invention can be administered by any of the conventional routes of administration. It should be noted that it can be administered as the compound or as pharmaceutically acceptable salt and can be administered alone or as an active ingredient in combination with pharmaceutically acceptable carriers, solvents, diluents, excipients, adjuvants and vehicles. The medicament or pharmaceutical composition can be administered orally, subcutaneously or parenterally including intravenous, intraarterial, intramuscular, intraperitoneally, and intranasal administration as well as intrathecal and infusion techniques. Implants of the medicament or pharmaceutical composition are also useful. Liquid forms may be prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles. In addition, under certain circumstances the compositions for use in the novel treatments of the present invention may be formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention.

When administering the medicament or pharmaceutical combination of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used have to be compatible with the compounds. Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques which deliver it orally or intravenously and retain the biological activity are preferred. In one embodiment, the compound of the present invention can be administered initially by intravenous injection to bring blood levels to a suitable level. The patient's levels are then maintained by an oral dosage form, although other forms of administration, dependent upon the patient's condition and as.indicated above, can be used.

In general, the active dose for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-2 weeks or longer, preferably for 24- to 48 hrs or by continuous infusion during a period of 1-2 weeks or longer.

It will be appreciated that the most appropriate administration of the pharmaceutical compositions of the present invention will depend on the type of injury or disease being treated. Thus, the treatment of an acute event will necessitate systemic administration of the active composition comparatively rapidly after induction of the injury. On the other hand, diminution of chronic degenerative damage may necessitate a sustained dosage regimen.

### Example 6

Preparation of Polypeptides

Polypeptides may be produced via several methods, for example:

1) Synthetically;

Synthetic polypeptides can be made using a commercially available machine, using the sequence of the sFRP1 polypeptide, as described in FIG. 1.

2) Recombinant Methods:

A preferred method of making sFRP1 is to clone the cDNA or a fragment thereof of the sFRP-1 gene, as described in FIG. 1, into an expression vector and culture the cell harboring the vector so as to express the encoded polypeptide, and then purify the resulting polypeptide, all performed using methods known in the art (Bibl Haematol. 1965;23:1165-74 Appl Microbiol. 1967 July;15(4):851-6; Can J. Biochem. 1968 May;46(5):441-4; Biochemistry. 1968 July;7(7):2574-80; Arch Biochem Biophys. 1968 Sep. 10;126(3):746-72, Biochem Biophys Res Commun. 1970 Feb. 20;38(4):825-30).

The expression vector can include a promoter for controlling transcription of the heterologous material and can be either a constitutive or inducible promoter to allow selective transcription. Enhancers that can be required to obtain necessary transcription levels can optionally be included. The expression vehicle can also include a selection gene.

Vectors can be made and subsequently introduced into cells or tissues by any one of a variety of methods known within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al. (1986).

3) Polypeptides (such as sFRP1) can be purified from natural sources (such as tissues or cultures stromal cells) using many methods known to one of ordinary skill in the art, such as for example: immuno-precipitation, or matrix-bound affinity chromatography with any molecule known to bind the desired polypeptide.

Protein purification is practiced as is known in the art as described in, for example, Marshak et al., “Strategies for Protein Purification and Characterization. A laboratory course manual.” CSHL Press (1996).

### Example 7

Preparation of Polynucleotides

The polynucleotides of the subject invention can be constructed by using a commercially available DNA synthesizing machine; overlapping pairs of chemically synthesized fragments of the desired gene can be ligated using methods well known in the art (e.g., see U.S. Pat. No. 6,121,426) and, for example, the nucleotide sequence described in FIG. 1.

Another means of isolating a polynucleotide is to obtain a natural or artificially designed DNA fragment based on that sequence. This DNA fragment is labeled by means of suitable labeling systems which are well known to those of skill in the art; see, e.g., Davis et al. (1986). The fragment is then used as a probe to screen a lambda phage cDNA library or a plasmid cDNA library using methods well known in the art; see, generally, Sambrook et al., Molecular Cloning: A Laboratory Manual, Cold Spring Harbor Laboratory Press, New York (1989), in Ausubel et al., Current Protocols in Molecular Biology, John Wiley and Sons, Baltimore, Md. (1989), Colonies can be identified which contain clones related to the cDNA probe and these clones can be purified by known methods. The ends of the newly purified clones are then sequenced to identify full-length sequences. Complete sequencing of full-length clones is performed by enzymatic digestion or primer walking.

A similar screening and clone selection approach can be applied to clones from a genomic DNA library.

### Example 8

Preparation of Antibodies

Antibodies which bind to the sFRP1 polypeptide may be prepared using an intact polypeptide or fragments containing smaller polypeptides as the immunizing antigen. For example, it may be desirable to produce antibodies that specifically bind to the N- or C-terminal or any other suitable domains of the sFRP1 polypeptide. The polypeptide used to immunize an animal can be derived from translated cDNA or chemical synthesis which can be conjugated to a carrier protein, if desired. Such commonly used carriers which are chemically coupled to the polypeptide include keyhole limpet hemocyanin (KLH), thyroglobulin, bovine serum albumin (BSA) and tetanus toxoid. The coupled polypeptide is then used to immunize the animal. Methods of immunization, including all necessary steps of preparing the immunogen in a suitable adjuvant, determining antibody binding, isolation of antibodies, methods for obtaining monoclonal antibodies, and humanization of monoclonal antibodies are all known to the skilled artisan

If desired, polyclonal or monoclonal antibodies can be further purified, for example by binding to and elution from a matrix to which the polypeptide or a peptide to which the antibodies were raised is bound. Those skilled in the art know various techniques common in immunology for purification and/or concentration of polyclonal as well as monoclonal antibodies (see, for example, Coligan et al, Unit 9, Current Protocols in Immunology, Wiley Interscience, 1994).

Methods for making antibodies of all types, including fragments, are known in the art (See for example, Harlow and Lane, Antibodies: A Laboratory Manual, Cold Spring Harbor Laboratory, New York (1988)). Methods of immunization, including all necessary steps of preparing the immunogen in a suitable adjuvant, determining antibody binding, isolation of antibodies, methods for obtaining monoclonal antibodies, and humanization of monoclonal antibodies are all known to the skilled artisan

The antibodies may be humanized antibodies or human antibodies. Antibodies can be humanized using a variety of techniques known in the art including CDR-grafting (EP239,400: PCT publication WO.91/09967; U.S. Pat. Nos. 5,225,539;5,530,101; and 5,585,089, veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,332).

The monoclonal antibodies as defined include antibodies derived from one species (such as murine, rabbit, goat, rat, human, etc.) as well as antibodies derived from two (or more) species, such as chimeric and humanized antibodies.

Completely human antibodies are particularly desirable for therapeutic treatment of human patients. Human antibodies can be made by a variety of methods known in the art including phage display methods using antibody libraries derived from human immunoglobulin sequences. See also U.S. Pat. Nos. 4,444,887 and 4,716,111; and PCT publications WO 98/46645, WO 98/50433, WO 98/24893, WO 98/16654, WO 96/34096, WO 96/33735, and WO 91/10741, each of which is incorporated herein by reference in its entirety.

Additional information regarding all types of antibodies, including humanized antibodies, human antibodies and antibody fragments can be found in WO 01/05998, which is incorporated herein by reference in its entirety.

### Example 9

Screening Systems

The sFRP1 gene or polypeptide may be used in a screening assay for identifying and isolating compounds which inhibit or stimulate stem cell proliferation. The compounds to be screened comprise inter alia substances such as small chemical molecules, antibodies, antisense oligonucleotides, antisense DNA or RNA molecules, polypeptides and dominant negatives, and expression vectors.

Many types of screening assays are known to those of ordinary skill in the art. The specific assay which is chosen depends to a great extent on the activity of the candidate gene or the polypeptide expressed thereby. Thus, if it is known that the expression product of a candidate gene has enzymatic activity, then an assay which is based on inhibition (or stimulation) of the enzymatic activity can be used. If the candidate polypeptide is known to bind to a ligand or other interactor, then the assay can be based on the inhibition of such binding or interaction. When the candidate gene is a known gene, such as sFRP1, then many of its properties can also be known, and these can be used to determine the best screening assay. If the candidate gene is novel, then some analysis and/or experimentation is appropriate in order to determine the best assay to be used to find inhibitors of the activity of that candidate gene. The analysis can involve a sequence analysis to find domains in the sequence which shed light on its activity.

As is well known in the art, the screening assays can be cell-based or non-cell-based. The cell-based assay is performed using eukaryotic cells such as HeLa cells, or possibly stem cells, and such cell-based systems are particularly relevant in order to directly measure the activity of candidate genes which are stem cell proliferation related genes, such as the sFRP1 gene. One way of performing such a cell-based assay involves the use of tetracycline-inducible (Tet-inducible) gene expression. Tet-inducible gene expression is well known in the art; see for example, Hofmann et al, 1996, Proc Natl Acad Sci 93(11):5185-5190.

Tet-inducible retroviruses have been designed incorporating the self-inactivating (SIN) feature of a 3′ Ltr enhancer/promoter retroviral deletion mutant. Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, thus indicating that expression is regulated uniformly within the infected cell population.

If the gene product of the candidate gene phosphorylates with a specific target protein, a specific reporter gene construct can be designed such that phosphorylation of this reporter gene product causes its activation, which can be followed by a color reaction. The candidate gene can be specifically induced, using the Tet-inducible system discussed above, and a comparison of induced versus non-induced genes provides a measure of reporter gene activation.

In a similar indirect assay, a reporter system can be designed that responds to changes in protein-protein interaction of the candidate protein. If the reporter responds to actual interaction with the candidate protein, a color reaction occurs.

One can also measure inhibition or stimulation of reporter gene activity by modulation of its expression levels via the specific candidate promoter or other regulatory elements. A specific promoter or regulatory element controlling the activity of a candidate gene is defined by methods well known in the art. A reporter gene is constructed which is controlled by the specific candidate gene promoter or regulatory elements. The DNA containing the specific promoter or regulatory agent is actually linked to the gene encoding the reporter. Reporter activity depends on specific activation of the promoter or regulatory element. Thus, inhibition or stimulation of the reporter is a direct assay of stimulation/inhibition of the reporter gene; see, for example, Komarov et al (1999), Science vol 285,1733-7 and Storz et al (1999) Analytical Biochemistry, 276, 97-104.

Various non-cell-based screening assays are also well within the skill of those of ordinary skill in the art. For example, if enzymatic activity is to be measured, such as if the candidate protein has a kinase activity, the target protein can be defined and specific phosphorylation of the target can be followed. The assay can involve either inhibition of target phosphorylation or stimulation of target phosphorylation, both types of assay being well known in the art; for example see Mohney et al (1998) J.Neuroscience 18, 5285 and Tang et al (1997) J. Clin. Invest. 100, 1180 for measurement of kinase activity. It is possible that sFRP1 interacts with an enzyme and regulates its enzymatic activity through protein-protein interaction.

One can also measure in vitro interaction of a candidate polypeptide with interactors. In this screen, the candidate polypeptide is immobilized on beads. An interactor, such as a receptor ligand, is radioactively labeled and added. When it binds to the candidate polypeptide on the bead, the amount of radioactivity carried on the beads (due to interaction with the candidate polypeptide) can be measured. The assay indicates inhibition of the interaction by measuring the amount of radioactivity on the bead.

Any of the screening assays, according to the present invention, can include a step of identifying the chemical compound (as described above) which tests positive in the assay and can also include the further step of producing as a medicament that which has been so identified. It is considered that medicaments comprising such compounds, or chemical analogs or homologs thereof, are part of the present invention. The use of any such compounds identified for inhibition or stimulation of stem cell proliferation, is also considered to be part of the present invention.

We claim:

1. A process for inducing proliferation of stem cells comprising administering to cultured stem cells sFRP1 polypeptide or an expression vector comprising the sFRP1 gene or a fragment thereof in a sufficient amount to cause proliferation of the stem cells.

2. The process of claim 1 wherein the stem cells are hematopoietic stem cells.

3. The process of claim 1 wherein the stem cells are embryonic stem cells.

4. A process for inducing proliferation of stem cells comprising culturing the stem cells with a second type of cells wherein the second type of cells express sFRP1 polypeptide.

5. The process of claim 4 wherein the expression is overexpression.

6. The process of claim 4 wherein the second type of cells are stromal cells.

7. The process of claim 4 further comprising administering sFRP1 polypeptide to the stem cells.

8. A method for treating a patient suffering from depletion of a cellular population comprising administering to the patient stem cells that have been expanded according to the method of claim 1.

9. A method for treating a patient suffering from depletion of a cellular population comprising administering to the patient stem cells that have been expanded according to the method of claim 4.

10. The method according to claim 8 wherein said patient suffers from depletion of a cellular population as a result of a disease or treatment thereof.

11. The method of claim 10 wherein the disease is cancer.

12. The method of claim 10 wherein the disease is a blood disorder.

13. The method of claim 10 wherein the disease is an auto-immune disease.

14. The method of claim 10 wherein the treatment comprises chemotherapy or radiotherapy.

15. A method for treating a patient suffering from depletion of a cellular population comprising administering to the patient a pharmaceutical composition comprising sFRP1 polypeptide or an expression vector comprising the sFRP1 gene or a fragment thereof, further comprising a pharmaceutically acceptable carrier in a dosage sufficient to induce proliferation of a cellular population.

16. The method according to claim 15 wherein said patient suffers from depletion of a cellular population as a result of a disease or treatment thereof.

17. The method of claim 16 wherein the disease is cancer.

18. The method according to claim 16 wherein the disease is a blood disorder.

19. The method according to claim 16 wherein the disease is an auto-immune disease.

20. The method of claim 16 wherein the treatment comprises chemotherapy or radiotherapy.

21. A pharmaceutical composition comprising sFRP1 polypeptide or an expression vector comprising the sFRP1 gene or a fragment thereof further comprising a pharmaceutically acceptable carrier.

22. A pharmaceutical composition according to claim 21 in a dosage sufficient to induce proliferation of a cellular population.

23. A process for identifying a compound which induces stem cell proliferation by modulation of sFRP1 polypeptide comprising:
(a) measuring the proliferative activity of the human sFRP1 polypeptide;
(b) contacting said polypeptide with said compound; and
(c) determining whether the activity of said polypeptide is affected by said compound.

24. A process of preparing a pharmaceutical composition which comprises the steps of:
(a) obtaining a compound by the process of claim 23; and
(b) admixing said compound with a pharmaceutically acceptable excipient.

25. A process for identifying a compound which induces stem cell proliferation by modulation of sFRP1 polypeptide comprising:
(a) measuring the binding of sFRP1 polypeptide to a species with which it interacts in vivo;
(b) contacting sFRP1 polypeptide with said compound; and
(c) determining whether the activity of sFRP1 polypeptide is affected by said compound.

26. A kit for identifying a compound which induces stem cell proliferation comprising:
(a) sFRP1 polypeptide;
(b) a species with which sFRP1 polypeptide interacts in vivo;
(c) means for measuring said interaction; and
(d) means for determining whether the binding of sFRP1 polypeptide to the species is affected by said compound.


SEQUENCE LISTING (2 sequences)

SEQ ID NO: 1 - DNA, 4469 bp, Homo sapiens
  CDS (303)..(1244)
cctgcagcctccggagtcagtgccgcgcgcccgccgccccgcgccttcctgctcgccgcacctccgggagccggggcgcacccagcccgcagcgccgcctccccgcccgcgccgcctccgaccgcaggccgagggccgccactggccggggggaccgggcagcagcttgcggccgcggagccgggcaacgctggggactgcgccttttgtccccggaggtccctggaagtttgcggcaggacgcgcgcggggaggcggcggaggcagccccgacgtcgcggagaacagggcgcagagccggcatgggcatcgggcgcagcgaggggggccgccgcggggccctgggcgtgctgctggcgctgggcgcggcgcttctggccgtgggctcggccagcgagtacgactacgtgagcttccagtcggacatcggcccgtaccagagcgggcgcttctacaccaagccacctcagtgcgtggacatccccgcggacctgcggctgtgccacaacgtgggctacaagaagatggtgctgcccaacctgctggagcacgagaccatggcggaggtgaagcagcaggccagcagctgggtgcccctgctcaacaagaactgccacgccgggacccaggtcttcctctgctcgctcttcgcgcccgtctgcctggaccggcccatctacccgtgtcgctggctctgcgaggccgtgcgcgactcgtgcgagccggtcatgcagttcttcggcttctactggcccgagatgcttaagtgtgacaagttcccggagggggacgtctgcatcgccatgacgccgcccaatgccaccgaagcctccaagccccaaggcacaacggtgtgtcctccctgtgacaacgagttgaaatctgaggccatcattgaacatctctgtgccagcgagtttgcactgaggatgaaaataaaagaagtgaaaaaagaaaatggcgacaagaagattgtccccaagaagaagaagcccctgaagttggggcccatcaagaagaaggacctgaagaagcttgtgctgtacctgaagaatggggctgactgtccctgccaccagctggacaacctcagccaccacttcctcatcatgggccgcaaggtgaagagccagtacttgctgacggccatccacaagtgggacaagaaaaacaaggagttcaaaaacttcatgaagaaaatgaaaaaccatgagtgccccacctttcagtccgtgtttaagtgattctcccgggggcagggtggggagggagcctcgggtggggtgggagcgggggggacagtgcccgggaacccgtggtcacacacacgcactgccctgtcagtagtggacattgtaatccagtcggcttgttcttgcagcattcccgctccctttccctccatagccacgctccaaaccccagggtagccatggccgggtaaagcaagggccatttagattaggaaggtttttaagatccgcaatgtggagcagcagccactgcacaggaggaggtgacaaaccatttccaacagcaacacagccactaaaacacaaaaagggggattgggcggaaagtgagagccagcagcaaaaactacattttgcaacttgttggtgtggatctattggctgatctatgcctttcaactagaaaattctaatgattggcaagtcacgttgttttcaggtccagagtagtttctttctgtctgctttaaatggaaacagactcataccacacttacaattaaggtcaagcccagaaagtgataagtgcagggaggaaaagtgcaagtccattatctaatagtgacagcaaagggaccaggggagaggcattgccttctctgcccacagtctttccgtgtgattgtctttgaatctgaatcagccagtctcagatgccccaaagtttcggttcctatgagcccggggcatgatctgatccccaagacatgtggaggggcagcctgtgcctgcctttgtgtcagaaaaaggaaaccacagtgagcctgagagagacggcgattttcgggctgagaaggcagtagttttcaaaacacatagttaaaaaagaaacaaatgaaaaaaattttagaacagtccagcaaattgctagtcagggtgaattgtgaaattgggtgaagagcttaggattctaatctcatgttttttccttttcacatttttaaaagaacaatgacaaacacccacttatttttcaaggttttaaaacagtctacattgagcatttgaaaggtgtgctagaacaaggtctcctgatccgtccgaggctgcttcccagaggagcagctctccccaggcatttgccaagggaggcggatttccctggtagtgtagctgtgtggctttccttcctgaagagtccgtggttgccctagaacctaacaccccctagcaaaactcacagagctttccgtttttttctttcctgtaaagaaacatttcctttgaacttgattgcctatggatcaaagaaattcagaacagcctgcctgttcccccgcactttttacatatatttgtttcatttctgcagatggaaagttgacatgggtggggtgtccccatccagcgagagagtttcaaaagcaaaacatctctgcagtttttcccaagtaccctgagatacttcccaaagcccttatgtttaatcagcgatgtatataagccagttcacttagacaactttacccttcttgtccaatgtacaggaagtagttctaaaaaaaatgcatattaatttcttcccccaaagccggattcttaattctctgcaacactttgaggacatttatgattgtccctctgggccaatgcttatacccagtgaggatgctgcagtgaggctgtaaagtggccccctgcggccctagcctgacccggagaaaggatggtagattctgttaactcttgaagactccagtatgaaaatcagcatgcccgcctagttacctaccggagagttatcctgataaattaacctctcacagttagtgatcctgtccttttaacaccttttttgtggggttctctctgacctttcatcgtaaagtgctggggaccttaagtgatttgcctgtaattttggatgattaaaaaatgtgtatatatattagctaattagaaatattctacttctctgttgtcaaactgaaattcagagcaagttcctgagtgcgtggatctgggtcttagttctggttgattcactcaagagttcagtgctcatacgtatctgctcattttgacaaagtgcctcatgcaaccgggccctctctctgcggcagagtccttagtggaggggtttacctggaacataagtagttaccacagaatacggaagagcaggtgactgtgctgtgcagctctctaaatgggaattctcaggtaggaagcaacagcttcagaaagagctcaaaataaattggaaatgtgaatcgcagctgtgggttttaccaccgtctgtctcagagtcccaggaccttgagtgtcattagttactttattgaaggttttagacccatagcagctttgtctctgtcacatcagcaatttcagaaccaaaagggaggctctctgtaggcacagagctgcactatcacgagcctttgtttttctccacaaagtatctaacaaaaccaatgtgcagactgattggcctggtcattggtctccgagagaggaggtttgcctgtgatttgcctgtgatttcctaattatcgctagggccaaggtgggatttgtaaagctttacaataatcattctggatagagtcctgggaggtccttggcagaactcagttaaatctttgaagaatatttgtagttatcttagaagatagcatgggaggtgaggattccaaaaacattttatttttaaaatatcctgtgtaacacttggctcttggtacctgtgggttagcatcaagttctccccagggtagaattcaatcagagctccagtttgcatttggatgtgtaaattacagtaatcccatttcccaaacctaaaatctgtttttctcatcagactctgagtaactggttgctgtgtcataacttcatagatgcaggaggctcaggtgatctgtttgaggagagcaccctaggcagcctgcagggaataacatactggccgttctgacctgttgccagcagatacacaggacatggatgaaattcccgtttcctctagtttcttcctgtagtactcctcttttagatcctaagtctcttacaaaagctttgaatactgtgaaaatgttttacattccatttcatttgtgttgtttttttaactgcattttaccagatgttttgatgttatcgcttatgttaatagtaattcccgtacgtgttcattttattttcatgctttttcagccatgtatcaatattcacttgactaaaatcactcaattaatcaatgaaaaaaaaaaa

SEQ ID NO: 2 - protein, 313 aa, Homo sapiens
MGIGRSEGGRRGALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK